(19) United States
(12) Patent Application Publication
(10) Pub. No.: US 2020/0172979 A1
(43) Pub. Date: Jun. 4, 2020

(54) COMPOSITIONS AND METHODS FOR IDENTIFYING CANCER

(71) Applicant: Zomedica Pharmaceuticals Corp., Ann Arbor, MI (US)

(72) Inventors: Casey J. Wegner, Ann Arbor, MI (US); Kevin Gorman, Ann Arbor, MI (US); Stephanie Morley, Ann Arbor, MI (US); Maithreyan Srinivasan, Palo Alto, CA (US); Ashley Wood, Ann Arbor, MI (US)

(21) Appl. No.: 16/389,706

(22) Filed: Apr. 19, 2019

Related U.S. Application Data
(60) Provisional application No. 62/775,160, filed on Dec. 4, 2018.

(51) Int. Cl. C12Q 1/6886; G16B 20/10; C12Q 1/6841

(52) U.S. Cl. CPC C12Q 1/6886; G16B 20/10; C12Q 1/6841; C12Q 2600/156; C12Q 2600/124; C12Q 2600/118; C12Q 2600/166

(57) ABSTRACT

Provided herein are compositions and methods for identifying cancer cells. In particular, provided herein are assays for identifying copy number variations (e.g., in circulating tumor cells (CTC)) indicative of cancer (e.g., lymphoma).

### FIELD OF THE DISCLOSURE

Provided herein are compositions and methods for identifying cancer cells. In particular, provided herein are assays for identifying copy number variations (e.g., in circulating tumor cells (CTC)) indicative of cancer (e.g., lymphoma).

### BACKGROUND OF THE DISCLOSURE

Over the decades pets moved from the yard to the house to the bed, becoming more and more like another family member every year. Pet owners' willingness to spend money on extending the lives of these precious family members has also increased, but there is a cap to the cost most owners are willing to pay when their pet has been diagnosed with cancer. Veterinary medicine is a largely cash-based business and requires the ability of the veterinarian, who is the advocate for their patient that cannot speak for itself, to show true value for the medical dollars spent and often maximize on minimal budgets.

Current tools for diagnosing cancer in companion animals are costly because they may require significant capital investment at the point of care (e.g., imaging modalities like ultrasound), surgical biopsy including anesthesia, surgeon time and post-op recovery, or histopathologic examination of the biopsy sample. Moreover, tissue biopsies are plagued by limitations such as invasiveness, lack of procedure repeatability on a patient, and inadequate diagnostic performance. Another problem with the diagnostic process for cancer patients is many animals suffering from cancer are not stable enough for surgical biopsy.

The development of cancer liquid biopsy tests, non-invasive blood testing alternatives to surgical biopsies, is an area of intense focus in human medicine. Cancer liquid biopsy approaches that primarily leverage circulating tumor DNA/RNA (ctDNA and ctRNA) or CTCs are increasingly being developed for use in diagnostic work-ups and screening in human medicine. However, liquid biopsy offerings have yet to take hold in veterinary medicine. This is likely attributed to a number of factors including cost constraints and a still limited amount of veterinary focused research investigations. A small handful of veterinary companies have developed blood-based cancer tests that rely on approaches such as ELISAs for inflammatory markers and whole blood mRNA signature panels. But these blood tests do not have the necessary diagnostic utility to be used as liquid biopsy tests.

Additional liquid biopsy tests for veterinary applications are needed.

### SUMMARY OF THE DISCLOSURE

Provided herein are compositions and methods for identifying cancer cells. In particular, provided herein are assays for identifying copy number variations (e.g., in circulating tumor cells (CTC)) or lymph tissue indicative of cancer (e.g., lymphoma). The compositions and methods described herein provide improved methods of diagnosing and characterizing lymphoma in blood and tissue samples. The methods described herein provide improved accuracy, decreased cost, and reduced time to diagnosis relative to existing methods.

For example, in some embodiments, the present disclosure provides a method of characterizing a sample from a subject, comprising: a) detecting the presence of a copy number variation in one or more regions (e.g., 1, 2, 3, 4, 5, or more regions) selected from those listed in Table 1 in the sample (e.g., using an oligo FISH assay); and b) characterizing the sample based on the presence of the copy number variations. In some embodiments, the characterizing comprises identifying the presence of lymphoma in the sample. In some embodiments, the characterizing comprises distinguishing between the presence of T cell lymphoma and B cell lymphoma in the sample. In some embodiments, the subject is a canine subject. The present disclosure is not limited to a particular sample types. Examples include but are not limited to, a tissue sample or a blood sample. In some embodiments, the sample is obtained by fine needle aspiration. In some embodiments, the blood sample comprises circulating tumor cells. In some embodiments, a gain in copy number of BOP1 and/or MYC regions and a loss in copy number in IGH and/or IGK regions is indicative of B cell lymphoma in the sample and a loss in copy number of the TP53 region is indicative of T cell lymphoma in the sample. In some embodiments, the copy number variations are variations relative to the level in a non-cancerous sample or a control region of the chromosome not subject to copy number variations. In some embodiments, the oligo FISH assay comprises a) contacting each of the regions with a plurality of labeled oligonucleotides specific for a different portion of the region and a plurality of oligonucleotides specific for a control region that is not subject to copy number variation; and b) comparing the number of labeled oligonucleotides bound to the region to the number of oligonucleotides bound to the control region. In some embodiments, the plurality of oligonucleotide comprises at least 2 (e.g., at least 2, 3, 4, 5, 10 or more) oligonucleotides per region. In some embodiments, the label is a fluorescent label. In some embodiments,

each of the plurality of oligonucleotides comprises a unique fluorescent barcode. In some embodiments, wherein said oligo FISH assay comprises a) contacting each of the regions with a plurality of labeled oligonucleotides specific for a different portion of the region, wherein each of the plurality of oligonucleotides comprises a unique fluorescent barcode; and b) determining the number of each unique fluorescent barcode in the sample. In some embodiments, the detecting comprises a multiplex assay.

Further embodiments provide a method of diagnosing lymphoma in a sample from a canine subject, comprising: a) detecting the presence of a copy number variation in one or more regions selected from those listed in Table 1 in the sample using an oligo FISH assay; and b) diagnosing lymphoma in the subject based on the presence of the copy number variations.

Additional embodiments provide the use of detecting the presence of a copy number variation in one or more regions selected from those listed in Table 1 (e.g., using an oligo FISH assay) in a sample from a subject to diagnose lymphoma in the subject.

Yet other embodiments provide a kit, comprising: a) a first plurality of labeled oligonucleotides that specifically bind to a first region selected from those listed in Table 1; and b) at least one second plurality of labeled oligonucleotides that specifically bind to a second region selected from those listed in Table 1.

Additional embodiments are described herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows A) traditional BAC-based FISH; and B) oligo-FISH of embodiments of the present disclosure.

FIG. 2 shows an exemplary work flow for oligo-FISH of embodiments of the present disclosure.

FIG. 3 shows B-cell vs. Normal CNVs. Each column represents a different probe, while the various regions are bounded by blue lines. Each sample is represented by a different color. High-frequency events are noted by a percentage. Data points above 0 indicate a gain, while points below 0 indicate a loss.

FIG. 4 shows T-cell vs. Normal CNVs. Each column represents a different probe, while the various regions are bounded by blue lines. Each sample is represented by a different color. High-frequency events are noted by a percentage. Data points above 0 indicate a gain, while points below 0 indicate a loss.

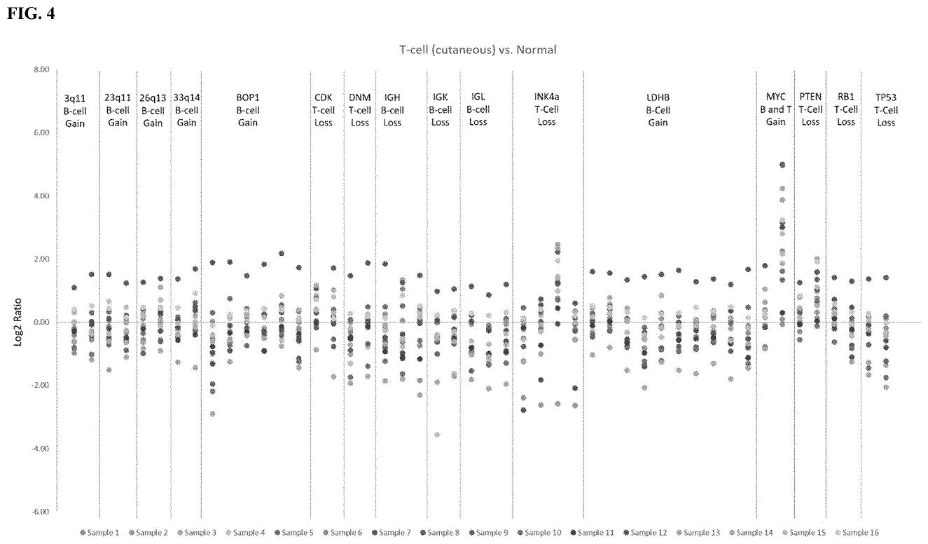

FIG. 5 shows B-cell vs. T-cell. Each column represents a different probe, while the various regions are bounded by blue lines. The average B-cell Log 2 ratio for a given probe is represented by blue dots, while the average T-cell Log 2 ratio is denoted by orange dots. Red boxes indicate candidate biomarkers that will be used for further development.

FIG. 6 shows representative images of canine-specific, oligoFISH probes staining nuclei of PBMCs derived from healthy canines.

FIG. 7 shows representative images of canine-specific, oligoFISH probes staining nuclei of PBMCs derived from confirmed canine lymphoma cases. Nuclei exhibiting CNV events are circled in green.

FIG. 8 shows a summary of CNVs identified from the group of diseased samples (7 total), and the single normal sample that was scored. For each probe set, 200 nuclei were examined by a technician and CNVs were determined according to the ratio of Target Probe:CEP Probe.

### DEFINITIONS

To facilitate an understanding of the present disclosure, a number of terms and phrases are defined below:

As used herein, the terms “detect”, “detecting”, or “detection” may describe either the general act of discovering or discerning or the specific observation of a composition.

The term “sample” as used herein is used in its broadest sense. As used herein, the term “sample” is used in its broadest sense. In one sense it can refer to a tissue sample. In another sense, it is meant to include a specimen or culture obtained from any source, as well as biological. Biological samples may be obtained from animals (including humans) and encompass fluids, solids, tissues, and gases. Biological samples include, but are not limited to blood products, such as plasma, serum and the like. These examples are not to be construed as limiting the sample types applicable to the present disclosure.

As used herein, the terms “complementary” or “complementarity” are used in reference to polynucleotides related by the base-pairing rules. For example, the sequence “5′-A-G-T-3′,” is complementary to the sequence “3′-T-C-A-S′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the term “nucleic acid molecule” refers to any nucleic acid containing molecule, including but not limited to, DNA or RNA. The term encompasses sequences that include any of the known base analogs of DNA and RNA including, but not limited to, 4 acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, 5-hydroxymethylcytosine, b-glucosyl-5-hydroxymethylcytosine, 5-formylcytosine, and 5-carboxycytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “nucleobase” is synonymous with other terms in use in the art including “nucleotide,” “deoxynucleotide,” “nucleotide residue,” “deoxynucleotide residue,” “nucleotide triphosphate (NTP),” or deoxynucleotide triphosphate (dNTP). An “oligonucleotide” refers to a nucleic acid that includes at least two nucleic acid monomer units (e.g., nucleotides), typically more than three monomer units, and more typically greater than ten monomer units. The exact size of an oligonucleotide generally depends on various factors, including the ultimate function or use of the oligonucleotide. To further illustrate, oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Typically, the nucleoside monomers are linked by phosphodiester bonds or analogs thereof, including phosphorothioate, phosphorodithioate, phosphoroselenoate, phosphorodiselenoate, phosphoroanilothioate, phosphoranilidate, phosphoramidate, and the like, including associated counterions, e.g., H⁺, NH₄⁺, Na⁺, and the like, if such counterions are present. Further, oligonucleotides are typically single-stranded. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (1979) Meth Enzymol. 68: 90-99; the phosphodiester method of Brown et al. (1979) Meth Enzymol. 68: 109-151; the diethylphosphoramidite method of Beaucage et al. (1981) Tetrahedron Lett. 22: 1859-1862; the triester method of Matteucci et al. (1981) J Am Chem Soc. 103:3185-3191; automated synthesis methods; or the solid support method of U.S. Pat. No. 4,458,066, entitled “PROCESS FOR PREPARING POLYNUCLEOTIDES,” issued Jul. 3, 1984 to Caruthers et al., or other methods known to those skilled in the art. All of these references are incorporated by reference.

A “sequence” of a biopolymer refers to the order and identity of monomer units (e.g., nucleotides, etc.) in the biopolymer. The sequence (e.g., base sequence) of a nucleic acid is typically read in the 5′ to 3′ direction.

As used herein, the term “subject” refers to any animal (e.g., a mammal), including, but not limited to, humans and companion animals (e.g., canines, felines, etc.), and the like, which is to be the recipient of a particular treatment. In some embodiments, the subject is a canine subject.

The term “gene” refers to a nucleic acid (e.g., DNA) sequence that comprises coding sequences necessary for the production of a polypeptide, RNA (e.g., including but not limited to, mRNA, tRNA and rRNA) or precursor. The polypeptide, RNA, or precursor can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) processed transcript. The mRNA functions during translation to specify the sequence or order of amino acids in a nascent polypeptide.

### DETAILED DESCRIPTION OF THE DISCLOSURE

Provided herein are compositions and methods for identifying cancer cells. In particular, provided herein are assays for identifying copy number variations (e.g., in circulating tumor cells (CTC)) indicative of cancer (e.g., lymphoma).

In companion animals (e.g., canines), lymphoma is typically diagnosed via fine needle aspiration (FNA) of organ lumps/masses. For canine lymphoma, FNA cytology is the primary means for initial diagnosis for general practitioners. In some instances, flow cytometry is performed by a reference lab ordered by veterinary oncologists to immunophenotype patient samples for suspected lymphoma cases. Patient sample preparation includes adding aspirate samples to a mixture of saline and patient serum. Other options for diagnosis are surgical biopsy cytology, immunohistochemistry (IHC) of biopsy samples or immunocytochemistry of FNA samples, and PCR for antigen receptor rearrangement (PARR). PARR is a clonality assay that helps to distinguish neoplastic from inflammatory lymphoid cells. Lymphoid neoplasms are monoclonal expansions of malignant lymphoid cells, whereas inflammatory lymphoid cells are usually polyclonal. Clonality is the hallmark of malignancy; PARR amplifies the variable regions of immunoglobulin genes and T-cell receptor genes to detect the presence of a clonal population.

Drawbacks of FNA of organ lumps/masses include subjective interpretation of results, proneness to sampling error, and subpar diagnostic accuracy and prognostic information. Immunophenotyping via flow cytometry for suspected canine lymphoma is overly expensive, slow (5-7 days for results), inaccurate as antibody targets are transiently expressed, and testing requires burdensome sample prep. Surgical biopsy is invasive, dangerous for patients, costly as surgery requires anesthesia, and interpretation of results is subjective. IHC immunophenotyping is prone to Ab cross-reactivity, sectioning errors, and sampling errors which impact. PARR is poorly diagnostic and prognostic for both B-cell and T-cell lymphomas when compared to Flow Cytometry or surgical biopsy cytology (B-cell sensitivity: 67%; T-cell sensitivity: 75%).

Taken together, these existing solutions do not offer a single test that can reliably deliver diagnostic and prognostic information. Nor do any of these test offer veterinarians and pet owners an affordable, quick and reliable means of diagnosing and immunophenotyping lymphoma in order to adequately inform treatment decision-making at the time of initial diagnosis.

Accordingly, provided herein are assays that uses copy number variation (CNV) (e.g., of blood samples) to both diagnose and immunophenotype cancer (e.g., lymphoma) in animals (e.g., human, canines, or other animals). Exemplary methods are described below.

### I. Detection of Copy Number Variations

Provided herein are assays for detecting copy number variations indicative of lymphoma and/or the immunophenotyped of lymphoma in a sample.

In some embodiments, CNVs are detected by assaying circulating tumor cells (CTCs) present in a blood or blood product sample. In some embodiments, CNVs in genomic DNA are detected in intact blood cells. In some embodiments, the sample is a tissue (e.g., biopsy) sample. In some embodiments, the sample is from a companion animal (e.g., canine).

In some embodiments, CNV detection methods utilize hybridization methods. In some embodiments, the hybridization is a fluorescence in situ hybridization (FISH) method. FISH is traditionally performed using fluorescently labeled DNA probes generated from known large chromosomal regions cloned into bacterial artificial chromosomes (BAC). These fluorescently labeled DNA probes are complementary to intended targets and hybridize. FISH is generally a single-cell technique that assesses the number of copies of targets present in every cell. Thus, deletions and amplifications result in the loss or gain of signal compared to control probes that are typically designed to centromeric regions.

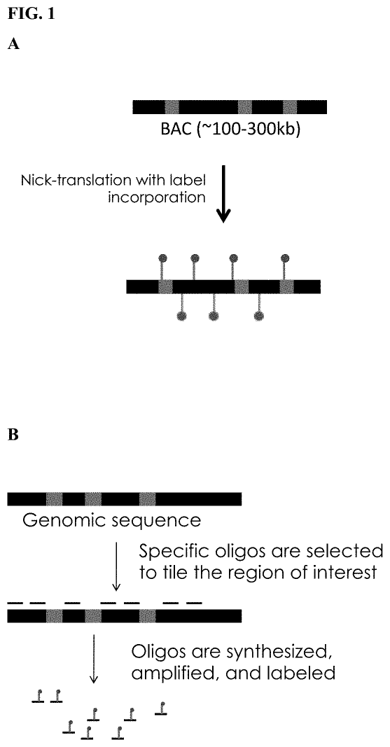

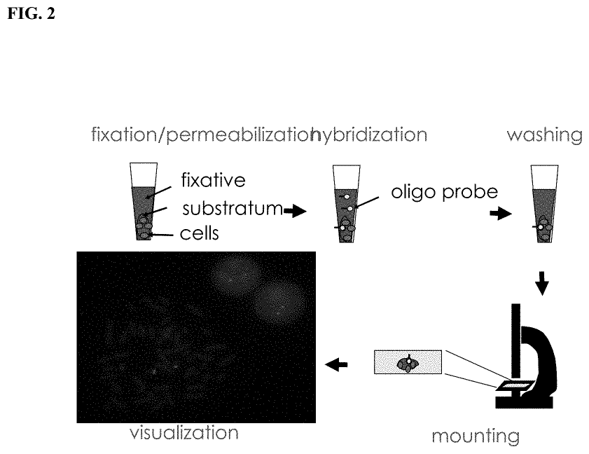

In some embodiments, provided herein are oligo FISH methods. Oligo FISH methods provide an advantage over traditional FISH that utilizes bacterial artificial chromosome (BAC) detection. For example, oligo FISH provides superior resolution, is customizable, and can detect small deletions or duplications that are difficult to detect with BAC based FISH. FIG. 1 compares BAC FISH (A) and oligo FISH (B). FIG. 2 shows an exemplary workflow for oligo FISH.

In some embodiments, oligo FISH uses a plurality (e.g., 2-50 (e.g., 2-40, 2-30 or 2-10)) of labeled oligonucleotides that tile the region of interest. In some embodiments, the oligonucleotides are 50-500 (e.g., 100-200) bp in length. In some embodiments, probes cover at least a portion of the region of interest (e.g., at least 1%, 5%, 10%, 20%, or 50%).

In some embodiments, assays detect one or more (e.g., 1, 2, 3, 4, 5, or more) regions of interest (e.g., those described in Table 1).

In some embodiments, the oligonucleotides comprise a fluorescent label. In some embodiments, a first set of oligonucleotide probes binds to the defined genomic area on the chromosomal DNA or the region of interest (Target Probe), while another oligonucleotide probe set (Control Probe) binds to a stable part of the same chromosome (e.g., not deleted or amplified). In some embodiments, the Target/Control probe ratio is calculated to determine if an amplification or deletion has occurred.

In some embodiments, a barcoded oligonucleotide assay is utilized. In some embodiments, oligonucleotides are designed specifically to recognize a portion of the genome and are tagged with a unique fluorescent barcode. Genomic DNA is prepared from the sample, incubated with the barcoded-oligonucleotides, and subsequently analyzed to determine how many times a given barcode was counted in the genomic DNA sample. By comparing the counts from disease and normal samples, one is able to generate a ratio to determine if an amplification or deletion has occurred within a specific genomic region. In some embodiments, commercially available bar coding and analysis assays (e.g., available from Nanostring, Seattle, Wash.) are used.

In some embodiments, assays for canine lymphoma comprise detection of CNVs in one or more chromosomal regions described in Table 1 to detect, diagnose and/or immunophenotype canine lymphoma. In some embodiments, a single assay described herein is able to both diagnose and immunophenotype (e.g., distinguish between T cell and B cell lymphoma) a sample. For example, in some embodiments, a gain in copy number of BOP1 and/or MYC regions and a loss in copy number in IGH and/or IGK regions is indicative of B cell lymphoma and a loss in copy number of the TP53 region is indicative of T cell lymphoma in the sample.

TABLE 1

| | Genomic Coordinates |
|---|---|
| Region Name | (CanFam 3.1) |
| IGHG | chr8: 72.95-74.12 (Mb) |
| MYC | chr13: 24.35-26.26 (Mb) |
| LDHB | chr13: 26.28-34.46 (Mb) |
| BOP1 | chr13: 37.44-38.22 (Mb) |
| IGKV6D-41 | chr17: 37.69-37.85 (Mb) |
| IGLV11-55 | chr26: 25.41-27.63 (Mb) |
| 3q34 | chr3: 74.56-74.61 (Mb) |
| 26q13 | chr26: 12.68-12.69 (Mb) |
| 23q11 | chr23: 0.92-0.95 (Mb) |
| 33q14 | chr33: 17.27-17.29 (Mb) |
| p16/INK4a | chr11: 40147753-43691621 |
| RB1 | chr22: 3053726-3204625 |
| PTEN | chr26: 37853148-37913176 |
| DNMT3A | chr17: 19489524-19563074 |
| CDKN1B | chr27: 33609154-33613558 |
| TP53 | chr5: 32561406-32565149 |

### II. Uses

As described herein, the present disclosure provides compositions and methods for detecting cancer cells in a sample. Such methods find use in research, screening, and diagnostic applications.

In some embodiments, the assays find use in diagnostic methods for identifying and characterizing cancer in a sample from a subject. In some embodiments, the subject is a non-human animal. In some embodiments, the non-human animal is a companion animal (e.g., dog, cat, etc.). The present disclosure is illustrated with canine samples. However, it is specifically contemplated that the described methods can be used to detect cancer cells in samples from other companion or non-companion animals.

In some embodiments, a computer-based analysis program is used to translate the raw data generated by the detection assay (e.g., the presence, absence, or amount of cancer marker) into data of predictive value for a clinician (e.g., presence of cancer or immunophenotype). The clinician can access the predictive data using any suitable means. Thus, in some preferred embodiments, the present disclosure provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present disclosure contemplates any method capable of receiving, processing, and transmitting the information to and from laboratories conducting the assays, information provides, medical personal, and subjects. For example, in some embodiments of the present disclosure, a sample (e.g., blood sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained (e.g., by a veterinary nurse) and sent to the profiling center, or subjects or pet owners may collect the sample themselves (e.g., a blood sample) and directly send it to a profiling center. Once received by the profiling service, the sample is processed and a profile is produced (e.g., cancer marker data), specific for the diagnostic or prognostic information desired for the subject.

The profile data is then prepared in a format suitable for interpretation by a treating clinician. For example, rather than providing raw data, the prepared format may represent a diagnosis (e.g., presence of cancer) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some exemplary embodiments, the sample (e.g., blood sample) is first obtained at the point of care (e.g., by a veterinary nurse), placed in a suitable container (e.g., vacuum blood tube), labeled with a unique identifier, and then sent to a testing lab (e.g., reference lab) by any suitable method. In some embodiments, the testing lab performs the analysis (e.g., using an automated system described herein) and provided results to the point of care provider in any suitable format (e.g., using an electronic portal). In some embodiments, depending on the analysis method, further sample preparation is performed at the point of care or testing laboratory (centrifugation).

In some exemplary embodiments, the sample (e.g., blood sample) is first obtained at the point of care (e.g., by a veterinary nurse), placed in a suitable container (e.g., cuvette), labeled with a unique identifier, and then sent to a testing lab (e.g., reference lab) by any suitable method. In some embodiments, the testing lab performs the analysis (e.g., using an automated system suitable for analysis of blood samples) and provided results to the point of care provider in any suitable format (e.g., using an electronic portal).

In some embodiments, all of the analysis is performed at the point of care (e.g., using an automated analysis system).

In some embodiments, the subject or pet owner is able to directly access the data using the electronic communication system. The subject or pet owner may choose further intervention or counseling based on the results. In some embodiments, the animal is treated with a therapeutic where the result indicates a particular disease stage (e.g., administered a chemotherapeutic agent or cocktail comprising, for example, one or more of doxorubicin, vinblastine, actinomycin-D, mitoxantrone, chlorambucil, methotrexate, DTIC, 9-aminocamptothecin, ifosfamide, cytosine, arabinoside, gemcitabine, lomustine, and dolastatin-10). In some embodiments, the data is used for research use. For example, the data may be used to further optimize the inclusion or elimination of markers as useful indicators of a particular condition or stage of disease.

### EXPERIMENTAL

The following examples are provided to demonstrate and further illustrate certain embodiments of the present disclosure and are not to be construed as limiting the scope thereof.

### Example 1

### Materials and Methods

### Disease Samples

Formalin-fixed paraffin embedded (FFPE) lymph-node tissue samples from confirmed cases of canine diffuse large B-cell lymphoma, and FFPE skin tissue samples from confirmed cases of epitheliotropic T-cell canine lymphoma were obtained from the College of Veterinary Medicine at Michigan State University.

### Healthy Controls

At the outset of this study, the goal was to obtain healthy lymph-node and skin FFPE samples for comparison with the B-cell and T-cell samples, respectively. However, normal tissue samples are rarely, if ever, banked for canine tissues. Thus, peripheral blood mononuclear cells (PBMCs) were purified from 10 dogs with no history of neoplasia and used as a control for non-cancerous cells.

### Nucleic Acid Extraction and Fragmentation

Genomic DNA extraction was performed at subsequently characterized for quality using a Nanodrop spectrometer. The gDNA was then fragmented according to the Nanostring protocol, and a QBIT analysis was performed to determine if the fragmentation was sufficient to proceed with Nanostring analysis.

### Nanostring Experiments to Identify CNVs

Sixteen potential regions for copy number variations for both B-cell and T-cell canine lymphomas were identified. Using the coordinates provided (CanFam 3.1), Nanostring designed probes to interrogate each potential region. The minimum number of probes per region was 2, while the larger regions received additional probes. The final probe designs can be found in Table 2.

Each 12-well cartridge (Nanostring) included two unique gDNA samples from normal animals, and a mixture of both B-cell and T-cell samples. In addition to the CNV probes, each well also contained positive and negative control probes.

TABLE 2
Nanostring Probe Design.

| | Genomic | | SEQ ID |
|---|---|---|---|
| ProbeID | Coordinates | Target Sequence | NO |
| 23q11_23856.1:25 | chr23:943383- | TCCTTGCACAAAGAATACCTAATTTGGCATGGGCCCAATCT | 1 |
| | 943465 | GCTTGCACCACCACCATCAGCCCGTCACTCCAAATGGCCTG | |
| | | CTCAGAAGCCCCGTGCAT | |
| 23q11_5084.1:189 | chr23:924773- | GTCCCATCATCTTTCATCTCCCAAATGTAGTGAGGATAATG | 2 |
| | 924872 | CTGCCTATTTCTGCTGACAATCTTCCTGAGAGACACTGTCCT | |
| | | CAAATTGCAAAATGTCC | |
| 26q13_2612.1:13 | chr26:12682125- | TTAGAGAACTTTCCTCTGAGAATGTCACCGACCACTATCAC | 3 |
| | 12682224 | AGACCCAGAGTGGGGATGAACGCTTCCTAACTTATTCGTAT | |
| | | AAAGGTGTATCTAAACTA | |
| 26q13_8405.1:134 | chr26:12688039- | GAATGAATGCACTGATGACAGCACTGTGTGCTTAGCACTCT | 4 |
| | 12688138 | CCTTATTCCCAACTTACTCGAGAAGAAACCAAGTCACAGAC | |
| | | AGAGGAAGGTTGGTCAAA | |
| 33q14_15050.1:190 | chr33:17284740- | TCAGAATGTTTTGGAACTTCTTTGGGATGATTACAAGAAAA | 5 |
| | 17284839 | CACAGAATGGATTCAAGTTTGTTTTCACCTGCCTGAGGCTG | |
| | | CTTTAATTTTATTTTTAT | |
| 33q14_4004.1:189 | chr33:17273693- | AAAAGCCTACCCCTAAGGATGGCACTTCATAGAAACATAA | 6 |
| | 17273792 | ATATAAAAAGCATGTGACACTTTCATTAAAAATAAGATGAC | |
| | | ATTCAGGTTGAGCAAGATG | |
| 3q34_14370.1:68 | chr3:74573938- | AGTTAACATCCACAAGAAGCATATACTTTAGGGCTCAGACC | 7 |
| | 74574037 | TGTTTGGACACAGCATGTTAAAAAAACAAAAAAAGAGGAT | |
| | | GGAGGCCTTGCACCTAAGA | |
| 3q34_36564.1:280 | chr3:74596344- | GGAGATGATTTGGATTTTTCTTGCCTTTCCTTCTACAAATTT | 8 |
| | 74596435 | TCTTCTGATACGATGTAAAGATGGTCCCCTAGGACCCAGCA | |
| | | GTGAGAGGCCAGCCAGG | |
| BOP1_272074.1:87 | chr13:37711675- | CCCACTCCCTTCCCCAGCGATGACACACACTCCAGCAGACA | 9 |
| | 37711745 | TGTGTCCCCAACCCCTACATCGAAATCGCCCTTCCAGCGCG | |
| | | GAGCGCGGCCCCTCACGG | |
| BOP1_279431.1:352 | chr13:37719298- | GAAGACAGGAGAACGGGAAGTGGGGACTGGCTGCTGGTTA | 10 |
| | 37719375 | GAAACAGGGCCCAGACCATCCAAAGAGTGAGTGTGAGCAG | |
| | | AAGACGTTGGGGAGAGTCCA | |
| BOP1_289981.1:22 | chr13:37729518- | CTTCTTGACCTGAAGGCCACGGAGTGATCAGAGTCACCAAC | 11 |
| | 37729588 | CTGCGTGTGTGTTCCTTCATCTGGAGATCCGTGCAGGGGCC | |
| | | AGTGGCCAGAGAGTAGGA | |
| BOP1_301899.1:88 | chr13:37741502- | GCCTCCCAGAACGTGGGTACCTCTCGGGACAAGGCCAAGG | 12 |
| | 37741573 | GGGTCAATTCCATGTCAGTGAGACATTCAGGAGGCGTGGCA | |
| | | AGGCCTGCTCACAGCCCCG | |
| BOP1_341927.1:179 | chr13:37781616- | CCTGCTCCAGGTCTTTCTCACTGAAGCCCTGGCCACTCAAGT | 13 |
| | 37781691 | CCAGTTCCCGCAGAGTGTGGCACCACTTCTGAGTCAACAGG | |
| | | TGGCTGCCCTCTTTGGC | |
| BOP1_343542.1:69 | chr13:37783123- | ACAGCCGCTGGCTTTCTACCAGCCGCCGACAGGAAGCCAGG | 14 |
| | 37783202 | GAAGAAAGAAAAGAGAGAAGGTCTCACCGATTGGGCATAA | |
| | | GCCACTCCAGGGAGGCGAG | |
| CDKN1B_2184.1:5 | chr27:33610843- | TTCATACTGATCAGATTTTAGTAGATGGAAAAAAATCTCCA | 15 |
| | 33610941 | CTTCTCCAGTGTGGGGTAGACCTACCCTCGCCAGCCCGTGT | |
| | | ATCTATTATACATTTCCC | |
| CDKN1B_3721.1:95 | chr27:33612482- | TCCGCTAGCCCCGTCTGGCTGTCGGGCGCATCAGTCTTTTGG | 16 |
| | 33612558 | TCTACCAAGTGTGTGTCCTCTGAGTTGGCCTGAGACCCCAG | |
| | | TAAAGGCCCCGCCTGGC | |
| DNMT3A_13120.1:8 | chr17:19502157- | GGGCAGCAATGCCACCCATCCAGGAAAGGCAGTAATTCGA | 17 |
| | 19502236 | GGACAAGAACCTGCCTATGAGAAGAGGGCCTGGACCCTGG | |
| | | GTGACTGGAGATTGTCCCTG | |
| DNMT3A_59707.1:218 | chr17:19548958- | ATTCTGAAGGCACCCGCTAGCCTGCTTCTCTCCTCACTTTGA | 18 |
| | 19549033 | TCAGACTCCAAGACACAGGTTCGCCCCTTCCAGGTCCTCAG | |
| | | GTGGGGCTGAGGCCCAG | |
| IGHG_1035498.1:28 | chr8:73985034- | TGTTTTGTCCTGATAACTGACATCGCTTCTGGGGCTGAGGTT | 19 |
| | 73985125 | CCTCTCCAAGATGCAGGGTCATGTCTGGGCTGTTTTATTCAA | |
| | | TAGAAGGTGGTCTCTA | |
| IGHG_608212.1:43 | chr8:73557761- | TGACTCTGCCCTGGAACTTCTGTGCATAACTTGTGGCACCAT | 20 |
| | 73557842 | CTTCAGGATCAATCTGTCCCATCCAATCAAGCCCTGCTCCTG | |
| | | GAGCCTGTCGTACCCA | |
| IGHG_72426.1:57 | chr8:73021990- | GGGCTCCTGGTTTTGAGCACAGGTCATGTCGCTGTTGGAGA | 21 |
| | 73022072 | TGGCATTCCAGTGCCCATAGTGACAGAGGCTTGGACAGTAT | |
| | | GGCCTGTCCCCAGAACTT | |
| IGKV6D41_158521.1:31 | chr17:37848052- | TTGCATCTTATCTAACCTCCCACTCTGACCTCTGGAGTCCCC | 22 |
| | 37848136 | TGTGGATTCAGGGACAGCGGACATGCTGCAGATACCTCACC | |
| | | CAGCCCGGATGTCAGGC | |
| IGKV6D41_8449.1:0 | chr17:37697949- | AGCTTGTTGTTCCATCTGCTAACAGATGGGAAGAACTCTGG | 23 |
| | 37698035 | CCTTAAATCTCAGTCTATTTCCAGGTGACCGGGCCATGCCA | |
| | | TCCAGCCTCTGCTCTTCA | |
| IGLV1155_1455721.1:3 | chr26:26512450- | TGCCCTCCCTCTCTGCATACCGGGGAACAAACTCCAGATGT | 24 |
| | 26512462 | ACCTACACCCTGAGCAGTGTCGCCAACTACTAAACATACTT | |
| | | CTCAAAGAGAATACAGGG | |
| IGLV1155_1618105.1:15 | chr26:25575728- | GGCCCAGATGAGTCTCTGGATCCAAGGATGCCTCCACTAAT | 25 |
| | 25575734 | ATAGCAATTCTGTGCATCTCTGGGGTGCAGCCTGAGGATGA | |
| | | GGGTGACTATGACTGTGC | |
| IGLV1155_1854915.1:151 | chr26:27264574- | AATTCCTCCCATGGAGCCAAACGTCCATGGATCAGGGAATC | 26 |
| | 27264660 | AGCATGTGAGCAGTAGGTTCAGAATCTTCACCTGGAGGAAG | |
| | | GTCTTCCTGCACCTGCAG | |
| INK4a_1077514.1:68 | chr11:41224847- | TCTGTTCGCTCACGCCCAACTAAGGTGCAGAGGGTCTAATG | 27 |
| | 41224934 | CCCCGATTCTGAGCCAACATGGGCTAGATCTCAGTATCTTC | |
| | | TCAGCCATGGTTTACCTT | |
| INK4a_1093711.1:87 | chr11:41241051- | GTGTTCAGTGTTTTTCCTGCATTCATTGCCACCCATCACAAT | 28 |
| | 41241150 | TCCTCAGTGTGCAGTTCAAATACCAGGTACAATGAGGACAC | |
| | | TATAGGCAGAAGTGAGG | |
| INK4a_1109435.1:219 | chr11:41256907- | ATAATAGGGAATGAGCAGATTCTAATTTTAGGCCTGGGGAC | 29 |
| | 41257006 | ATTTCACAAATCCCATCAGTATGTAGACCATGTAAGTTTGTT | |
| | | TTGGTTTGATTTGATTA | |
| INK4a_1117751.1:1 | chr11:41265005- | GCTAAGGTTTTGGAAAAGTTGAAGCAACGCGCTCAGTTCCT | 30 |
| | 41265089 | AATCCCCTCCCTCCAGCAGGCGGAGCAGAGGGTTTCTGTTC | |
| | | CTTGAGCCCCGTGCCGGC | |
| LDHB_1072854.1:334 | chr13:27352688- | GTATTATTAGAGTTAGGCTGATAAGGGTACATAAAGCACAG | 31 |
| | 27352783 | AGCAAAATGCTGGGTGCTCCACAATGTACGGTAAACAAGG | |
| | | AGGTATCTCCCCTCTGGCC | |
| LDHB_1625806.1:136 | chr13:27905442- | CAAGTCTCAAGTAGGATCTATTTGAGATTCCAATGGCAAAT | 32 |
| | 27905541 | GAGACAACTAACATTCCATCCCTACGAGGCATCCTGTGGTG | |
| | | ACTTTAATTTAGATCAAG | |
| LDHB_211424.1:45 | chr13:26490969- | GGCTGTTCATGGTAATCCAAGGAAGTGGTTGGAGTGAATTC | 33 |
| | 26491068 | TACAATTCTTTGGCCTTTAAGTCCTGAAGATGACTAGTTAA | |
| | | ATAATTTGATGCACAGAC | |
| LDHB_2734456.1:32 | chr13:29013988- | TGGCAAAGGAGAAGAGACAAGTAGCATGATCCTTTTAGGA | 34 |
| | 29014087 | GAGGAATATAAATATAAGTCAGCAAGAAGGAAAAAAAAAG | |
| | | ATATATCGAGGGGAGAAGAC | |
| LDHB_3447136.1:18 | chr13:29726654- | CCTGGGCTACGGTGAGTGTTTTGATTCAGAAAGGGGGAAAA | 35 |
| | 29726753 | AAAAATCTTGACTTCTTTGGGAAAAGAACAAAGCGTCTGCT | |
| | | TGAGTTACTCAAAACGCT | |
| LDHB_4418467.1:104 | chr13:30698071- | ACTGCTGCTCTCCAGTAGGGAAATCCACCTGGTAAATCTAT | 36 |
| | 30698165 | TTCCAACCCCTTACCTGGGCAGGACAAGCACTTTCAAGGAT | |
| | | GCAATGGGTTACGCAGCC | |
| LDHB_4940278.1:53 | chr13:31219831- | TTTCAATATCTGATTCCTTTCTTTTGTTCCCTCTTCACCAACC | 37 |
| | 31219922 | CACTCCTGATGCCCAAGATCTGAGCACCAGAAGAGTGGAG | |
| | | AAGGTGGTGGAAGGGCA | |
| LDHB_5823105.1:137 | chr13:32102742- | AATACAGCATAGTGTATCACCATGTAGTAGATGAAGGATGT | 38 |
| | 32102841 | ATCAGGGCACCAGGACACCACAAGAATATAGCCAGTCTAC | |
| | | AACTGGGACTGTATTATTT | |
| LDHB_6829110.1:98 | chr13:33108708- | CAGTAATGGCAGAAATGAAGTCATTTATAGATGCTCGTTAC | 39 |
| | 33108807 | TATGAACACATGCATCTTCTACAGTCATGAGTGGATTTCTTA | |
| | | CACATATCTAATCCAGT | |
| LDHB_7688219.1:79 | chr13:33967813- | CTGCCGGCCCTGGAGAGCGCTGTTACCAAACACCGGTCGTG | 40 |
| | 33967891 | ACCCAGGACGGACAGATGTCACCCCAATGAAGCAAGCAGA | |
| | | ACAGAGGTTGGACACAGGC | |
| MYC_852192.1:2 | chr13:25201699- | CTCCGCAGCCGCGGACTTTTCCGCGTCTCCGAAAGGGTATT | 41 |
| | 25201781 | TAAATTTCATCTTTACCGCATTTCTGACAGCCGGAGGCAGA | |
| | | CACTGCGGCGCGTCCCGC | |
| MYC_855107.1:368 | chr13:25204986- | GTGGTAGAGTCCTGAAACAGATCAGCAACAACCGCAAATG | 42 |
| | 25205065 | TGCCAGCCCCAGGTCTTCGGACACGGAGGAGAATGACAAG | |
| | | AGGCGAACACACAACGTCTT | |
| PTEN_3964.1:53 | chr26:37856665- | AGTGAACTGGCTTTCTTACTGCAGAACCTTTAATACACTGCT | 43 |
| | 37856764 | GTGTCTTGTGAATCTCTCAAGAGTGAAATTCGAATATGCTG | |
| | | CTTTCCTGTTCCTTTTT | |
| PTEN_51429.1:46 | chr26:37904123- | AGAGAGAGAGATGTGGTATTTTGGAAAGCACCAGGCAACA | 44 |
| | 37904219 | GACAAAGTAGTAACTAGCCTTCTGTGTTAATCAGGTACGGG | |
| | | GCTAGAGGGTGGGAAGGGA | |
| RB1_120766.1:65 | chr22:3174057- | GGTTGTTATGATAGGCATACACATTTATTTCTACACCTACAT | 45 |
| | 3174156 | GTTTTGAAAATCTTAAGTTCACATCCATTTATCCAATTCCAA | |
| | | CATAAAACCACATGGT | |
| RB1_21414.1:224 | chr22:3074864- | TATCAAAGGTTCAAACTATCAGATAAAATACTGATGGTGGT | 46 |
| | 3074963 | AAGGAGTTTGGAAGGGGGCAGGAGAGAGTCTTTGTATTGT | |
| | | AGGTAATATTCTTGATTTG | |
| TP53_1254.1:67 | chr5:32562227- | GGGGGGAGGAGGAGAAAGAATAAGAGGGGGAAGCCACAG | 47 |
| | 32562318 | TACCATTTAACTCAGACTAAGGTGTAAGTGCCAATCTGCAG | |
| | | CAAGGCACAGCCCGCAGCCA | |
| TP53_3509.1:9 | chr5:32564424- | CAAAGGTCACAGAATACATAGGTAATGCAGGAAGACTGAC | 48 |
| | 32564516 | AGTGCGGACCGAGAGGAGTCTCAAAATCCCAAACAGAGAA | |
| | | ACCCAGGGGCCACAGCCAGG | |
| VEGFA_12413.1:75 | chr12:12221035- | GCCTGGCGGGGCCTGTTCAGAGGTTGCCCTGGTTGCCTGAG | 49 |
| | 12221104 | GGGGTAGACTGGTGTGGCTTCATGTGATGGTGTGGACGCAG | |
| | | GCTGAGTGTTGTGTCCTG | |
| VEGFA_3177.1:121 | chr12:12211830- | TGTGGGTTTGCTGAATATACCTGAGGATATTTGCTGAGTATT | 50 |
| | 12211914 | CCGGAGGCCAGAGGAGTTTGGGTGGGGGAGGGTGGTTGGT | |
| | | GTCCTTCGGTCCTCCGCA | |

### Data Analysis

The RCC files from each run were loaded into the nSolver 4.0 software (Nanostring) for analysis. Each cartridge was run with gDNA from two normal animals. A normalization factor was created for each sample based on the counts from the invariant control region (e.g. VEGFA). The normalized counts for each sample were used to create ratios between the disease sample and the average count for both normal samples in a given cartridge. The ratios were transformed using the Log 2 function, and plotted using Microsoft Excel.

### FISH Probe Design and Manufacturing

FISH probes were designed for the following regions: TP53 (CEPS), IGH (CEPS), IGK (CEP17), BOP1 and MYC (both use CEP13). Probe designs, coverage size, and oligo # for each probe set can be found in Table 3.

TABLE 3
oligoFISH probe designs

| | Probe Region | Coverage | % | # of |
|---|---|---|---|---|
| Probe | (CanFam2) | Size | Coverage | Oligos |
| FISH CFA TP53 | chr5: 35499791- | 124418 | 80 | 17009 |
| 124 kb | 35624209 | | | |
| FISH CFA MYC | chr13: 27930129- | 650629 | 78 | 84126 |
| 651 kb | 28580758 | | | |
| FISH CFA IGH | chr8: 75940536- | 394737 | 45 | 24257 |
| 395 kb | 76335273 | | | |
| FISH CFA IGK | chr17: 40617324- | 258541 | 49 | 20789 |
| 259 kb | 40875865 | | | |
| FISH CFA Chr17 | chr17: 3003005- | 613066 | 76 | 83146 |
| CEP 613 kb | 3616071 | | | |
| FISH CFA Chr8 | chr8: 3608695- | 601731 | 47 | 43973 |
| CEP 602 kb | 4210426 | | | |
| FISH CFA Chr5 | chr5: 3046245- | 595720 | 59 | 54689 |
| CEP 596 kb | 3641965 | | | |
| FISH CFA Chr13 | chr13: 3029325- | 618731 | 72 | 74001 |
| CEP 619 kb | 3648056 | | | |

### FISH Staining Protocol

PBMCs were isolated from whole blood and fixed using Carnoy's fixative. Fixed cells were then stored in the freezer for at least 30 minutes. Next, fixed cells were added to microscope slides and were warmed to 45° C. for 15 minutes, and subsequently cooled to RT. Slides were then dehydrated using ethanol, and the FISH probes were added directly to the slides for hybridization. Hybridization was performed using the following parameters: 90° for 5 minutes, 45° C. for 90 minutes. Slides were washed, counterstained with DAPI, and a coverslip was added for subsequent analysis on the microscope.

FISH imaging and Analysis

A Zeiss Axio Imager M2 microscope was used to visualize stained PBMCs. Exposure time was variable; it was adjusted automatically by the software (MetaSystems) based on signal intensity. Images were recorded with a CCD camera (MetaSystems) and subsequently analyzed by a technician. Ratios of each color appearing in a given nuclei were scored by the technician and reported.

### Results

### CNVs in B-Cell Tissue Samples

Genomic microarrays were used to identify potential CNVs from splenic tissue samples. Gains or losses are demonstrated in certain regions.

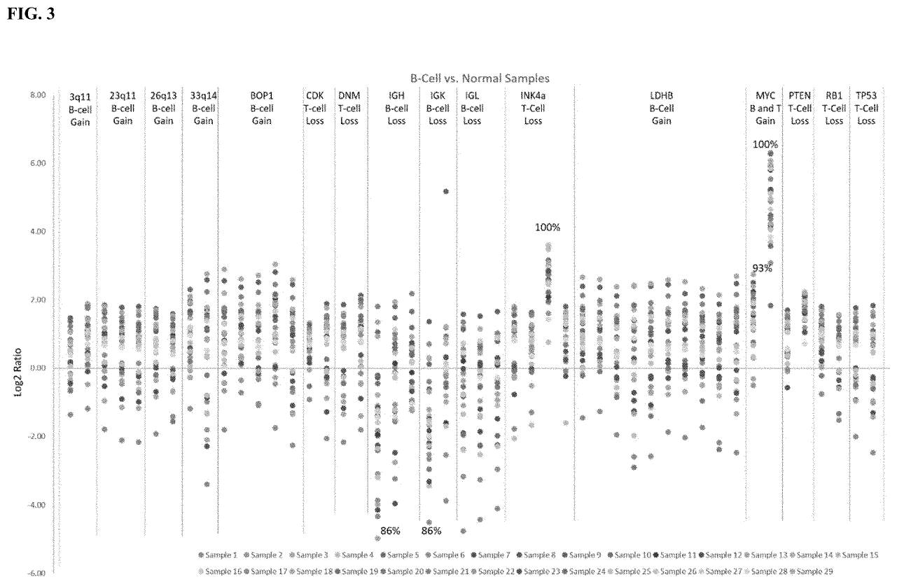

FIG. 3 is a plot of the Log 2 ratio between the B-cell sample count and the average normal count for each probe. Values above 0 indicate a gain event, while those below 0 indicate a loss event. Each region is denoted in text at the top of the graph, and indicate the expected sample type and expected CNV event (gain or loss). The most frequent gains in the B-cell population were observed in the BOP1 and MYC regions, while the most common losses occurred in the IGH and IGK regions.

The INK4A region showed an amplification in all samples, despite this being an expected loss only in T-cell cancers according to the literature. The data from the cutaneous T-cell samples also indicates an amplification, indicated that this gain is irrespective of immunophenotype.

### CNVs in T-Cell Tissue Samples

TP53 indicated a frequent loss event. Myc is amplified in T-cell as well as B-cell samples.

### Comparing B-Cell and T-Cell CNVs in Tissue

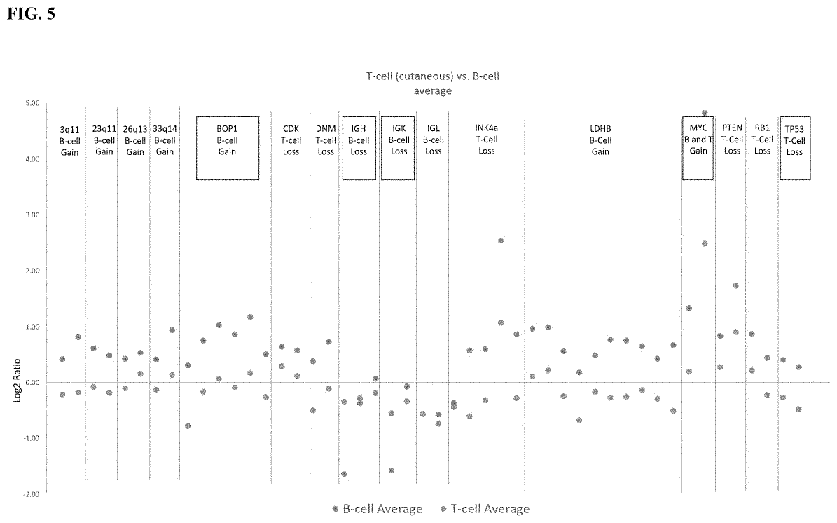

The next step was to compare B-cell and T-cell CNVs to determine CNVs that could be used to distinguish one another. To do this, the average Log 2 value for each probe in a given patient population were plotted on the same graph (FIG. 5).

CNVs that were able to differentiate between B-cell and T-cell patients when compared against healthy controls were identified. Using these criteria, 7 CNVs were identified: BOP1, IGH, IGK, MYC, INK4a, LDHB and TP53. The BOP1 region shows a clear amplification in B-cell samples, while the T-cell samples are in the normal range (around 0). In the IGH and IGK regions, there are profound deletion events in B-cell samples while again, T-cell samples are in the normal range. MYC shows a frequent amplification in both B-cell and T-cell samples, and is therefore useful as a positive control region. Finally, the TP53 region shows a slight loss in T-cell samples and a slight gain in B-cell samples.

### FISH Staining in Normal Blood Samples

Reagents were tested in a FISH assay using canine PBMCs from normal samples. Experiments with normal samples showed that each probe set gave good signal intensity and appeared to bind to the proper region of the genome.

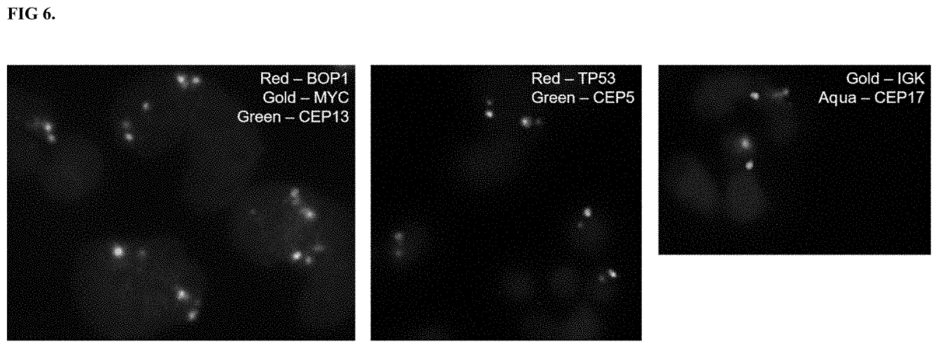

In this assay setup, CNVs are apparent based on the ratio of a given target probe to its CEP (control) probe (FIG. 6).

### FISH Staining in Canine Lymphoma Blood Samples

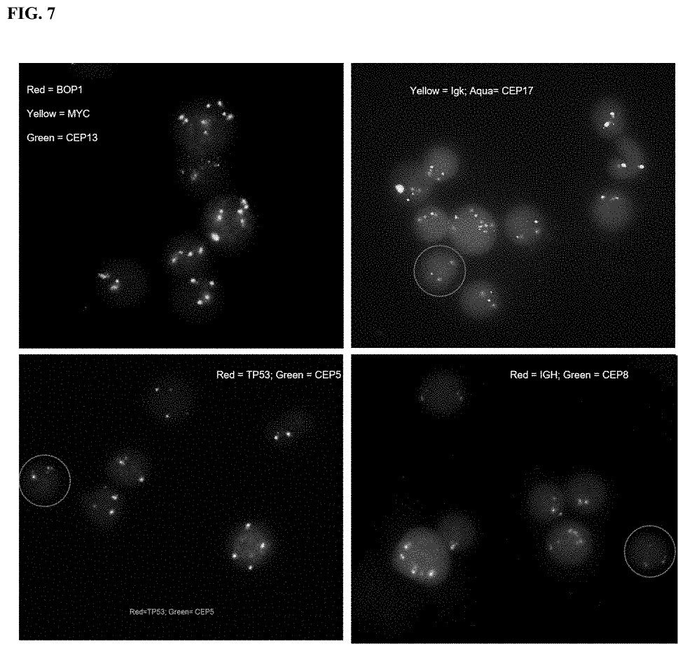

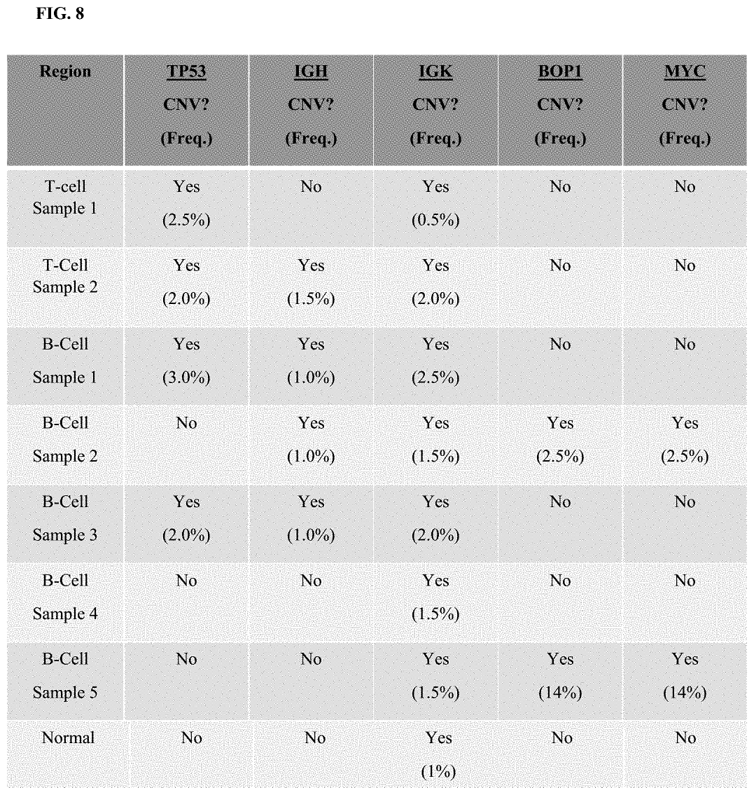

The probes were tested in disease samples. For this set of experiments, PBMCs from confirmed cases of B-cell or T-cell lymphoma were subjected to FISH using each set of probes. Two-hundred nuclei from each sample were scored for each probe set to determine the frequency of CNV events. Results are shown in FIG. 7 and the Table shown in FIG. 8. A CNV was found in all of the samples for the IGK region, to varying degrees of frequency. The CNV in the IGK region was also detected in the one normal sample that was scored. These studies are based on the underlying frequency of a given CNV.

All publications, patents, patent applications and accession numbers mentioned in the above specification are herein incorporated by reference in their entirety. Although the disclosure has been described in connection with specific embodiments, it should be understood that the disclosure as claimed should not be unduly limited to such specific embodiments. Indeed, various modifications and variations of the described compositions and methods of the disclosure will be apparent to those of ordinary skill in the art and are intended to be within the scope of the following claims.

We claim:

1. A method of characterizing a sample from a canine subject, comprising:
a) detecting the presence of a copy number variation in two or more regions selected from those listed in Table 1 in said sample, wherein said regions include two or more of BOP1, MYC, IGH, IGK and TP53; and
b) characterizing said sample based on the presence of said copy number variations.

2. The method of claim 1, wherein said characterizing comprises identifying the presence of lymphoma in said sample.

3. The method of claim 1, wherein said characterizing comprises distinguishing between the presence of T cell lymphoma and B cell lymphoma in said sample.

4. The method of claim 1, wherein said detecting comprises an oligo FISH assay.

5. The method of claim 1, wherein said sample is selected from the group consisting of a tissue sample and a blood sample.

6. The method of claim 5, wherein said blood sample comprises circulating tumor cells.

7. The method of claim 1, wherein a gain in copy number of BOP1 and/or MYC regions and a loss in copy number in IGH and/or IGK regions is indicative of B cell lymphoma in said sample.

8. The method of claim 1, wherein a loss in copy number of the TP53 region is indicative of T cell lymphoma in said sample.

9. The method of claim 1, wherein said copy number variations are variations relative to the level in a non-cancerous sample.

10. The method of claim 4, wherein said oligo FISH assay comprises a) contacting each of said regions with a plurality of labeled oligonucleotides specific for a different portion of said region and a plurality of oligonucleotides specific for a control region that is not subject to copy number variation; and b) comparing the number of labeled oligonucleotides bound to said region to the number of oligonucleotides bound to said control region.

11. The method of claim 10, wherein said plurality of oligonucleotide comprises at least 2 oligonucleotides per region.

12. The method of claim 10, wherein said label is a fluorescent label.

13. The method of claim 12, wherein each of said plurality of oligonucleotides comprises a unique fluorescent barcode.

14. The method of claim 4, wherein said oligo FISH assay comprises a) contacting each of said regions with a plurality of labeled oligonucleotides specific for a different portion of said region, wherein each of said plurality of oligonucleotides comprises a unique fluorescent barcode; and b) determining the number of each unique fluorescent barcode in said sample.

15. A method of diagnosing lymphoma in a sample from a canine subject, comprising:
a) detecting the presence of a copy number variation in two or more regions selected from those listed in Table 1, wherein said regions include two or more of BOP1, MYC, IGH, IGK and TP53; and
b) diagnosing lymphoma in said subject based on the presence of said copy number variations.

16. The method of claim 15, wherein a gain in copy number of BOP1 and/or MYC regions and a loss in copy number in IGH and/or IGK regions is indicative of B cell lymphoma in said sample.

17. The method of claim 15, wherein a loss in copy number of the TP53 region is indicative of T cell lymphoma in said sample.

18. The method of claim 1, wherein said sample comprises circulating tumor cells.

19. A kit, comprising:
a) a first plurality of labeled oligonucleotides that specifically bind to a first region of targets selected from those listed in Table 1; and
b) a second plurality of labeled oligonucleotides that specifically bind to a second region of targets selected from those listed in Table 1.


SEQUENCE LISTING (50 sequences)

SEQ ID NO: 1 - DNA, 100 bp, Artificial sequence
  Synthetic
tccttgcacaaagaatacctaatttggcatgggcccaatctgcttgcaccaccaccatcagcccgtcactccaaatggcctgctcagaagccccgtgcat

SEQ ID NO: 2 - DNA, 100 bp, Artificial sequence
  Synthetic
gtcccatcatctttcatctcccaaatgtagtgaggataatgctgcctatttctgctgacaatcttcctgagagacactgtcctcaaattgcaaaatgtcc

SEQ ID NO: 3 - DNA, 100 bp, Artificial sequence
  Synthetic
ttagagaactttcctctgagaatgtcaccgaccactatcacagacccagagtggggatgaacgcttcctaacttattcgtataaaggtgtatctaaacta

SEQ ID NO: 4 - DNA, 100 bp, Artificial sequence
  Synthetic
gaatgaatgcactgatgacagcactgtgtgcttagcactctccttattcccaacttactcgagaagaaaccaagtcacagacagaggaaggttggtcaaa

SEQ ID NO: 5 - DNA, 100 bp, Artificial sequence
  Synthetic
tcagaatgttttggaacttctttgggatgattacaagaaaacacagaatggattcaagtttgttttcacctgcctgaggctgctttaattttatttttat

SEQ ID NO: 6 - DNA, 100 bp, Artificial sequence
  Synthetic
aaaagcctacccctaaggatggcacttcatagaaacataaatataaaaagcatgtgacactttcattaaaaataagatgacattcaggttgagcaagatg

SEQ ID NO: 7 - DNA, 100 bp, Artificial sequence
  Synthetic
agttaacatccacaagaagcatatactttagggctcagacctgtttggacacagcatgttaaaaaaacaaaaaaagaggatggaggccttgcacctaaga

SEQ ID NO: 8 - DNA, 100 bp, Artificial sequence
  Synthetic
ggagatgatttggatttttcttgcctttccttctacaaattttcttctgatacgatgtaaagatggtcccctaggacccagcagtgagaggccagccagg

SEQ ID NO: 9 - DNA, 100 bp, Artificial sequence
  Synthetic
cccactcccttccccagcgatgacacacactccagcagacatgtgtccccaacccctacatcgaaatcgcccttccagcgcggagcgcggcccctcacgg

SEQ ID NO: 10 - DNA, 100 bp, Artificial sequence
  Synthetic
gaagacaggagaacgggaagtggggactggctgctggttagaaacagggcccagaccatccaaagagtgagtgtgagcagaagacgttggggagagtcca

SEQ ID NO: 11 - DNA, 100 bp, Artificial sequence
  Synthetic
cttcttgacctgaaggccacggagtgatcagagtcaccaacctgcgtgtgtgttccttcatctggagatccgtgcaggggccagtggccagagagtagga

SEQ ID NO: 12 - DNA, 100 bp, Artificial sequence
  Synthetic
gcctcccagaacgtgggtacctctcgggacaaggccaagggggtcaattccatgtcagtgagacattcaggaggcgtggcaaggcctgctcacagccccg

SEQ ID NO: 13 - DNA, 100 bp, Artificial sequence
  Synthetic
cctgctccaggtctttctcactgaagccctggccactcaagtccagttcccgcagagtgtggcaccacttctgagtcaacaggtggctgccctctttggc

SEQ ID NO: 14 - DNA, 100 bp, Artificial sequence
  Synthetic
acagccgctggctttctaccagccgccgacaggaagccagggaagaaagaaaagagagaaggtctcaccgattgggcataagccactccagggaggcgag

SEQ ID NO: 15 - DNA, 100 bp, Artificial sequence
  Synthetic
ttcatactgatcagattttagtagatggaaaaaaatctccacttctccagtgtggggtagacctaccctcgccagcccgtgtatctattatacatttccc

SEQ ID NO: 16 - DNA, 100 bp, Artificial sequence
  Synthetic
tccgctagccccgtctggctgtcgggcgcatcagtcttttggtctaccaagtgtgtgtcctctgagttggcctgagaccccagtaaaggccccgcctggc

SEQ ID NO: 17 - DNA, 100 bp, Artificial sequence
  Synthetic
gggcagcaatgccacccatccaggaaaggcagtaattcgaggacaagaacctgcctatgagaagagggcctggaccctgggtgactggagattgtccctg

SEQ ID NO: 18 - DNA, 100 bp, Artificial sequence
  Synthetic
attctgaaggcacccgctagcctgcttctctcctcactttgatcagactccaagacacaggttcgccccttccaggtcctcaggtggggctgaggcccag

SEQ ID NO: 19 - DNA, 100 bp, Artificial sequence
  Synthetic
tgttttgtcctgataactgacatcgcttctggggctgaggttcctctccaagatgcagggtcatgtctgggctgttttattcaatagaaggtggtctcta

SEQ ID NO: 20 - DNA, 100 bp, Artificial sequence
  Synthetic
tgactctgccctggaacttctgtgcataacttgtggcaccatcttcaggatcaatctgtcccatccaatcaagccctgctcctggagcctgtcgtaccca

SEQ ID NO: 21 - DNA, 100 bp, Artificial sequence
  Synthetic
gggctcctggttttgagcacaggtcatgtcgctgttggagatggcattccagtgcccatagtgacagaggcttggacagtatggcctgtccccagaactt

SEQ ID NO: 22 - DNA, 100 bp, Artificial sequence
  Synthetic
ttgcatcttatctaacctcccactctgacctctggagtcccctgtggattcagggacagcggacatgctgcagatacctcacccagcccggatgtcaggc

SEQ ID NO: 23 - DNA, 100 bp, Artificial sequence
  Synthetic
agcttgttgttccatctgctaacagatgggaagaactctggccttaaatctcagtctatttccaggtgaccgggccatgccatccagcctctgctcttca

SEQ ID NO: 24 - DNA, 100 bp, Artificial sequence
  Synthetic
tgccctccctctctgcataccggggaacaaactccagatgtacctacaccctgagcagtgtcgccaactactaaacatacttctcaaagagaatacaggg

SEQ ID NO: 25 - DNA, 100 bp, Artificial sequence
  Synthetic
ggcccagatgagtctctggatccaaggatgcctccactaatatagcaattctgtgcatctctggggtgcagcctgaggatgagggtgactatgactgtgc

SEQ ID NO: 26 - DNA, 100 bp, Artificial sequence
  Synthetic
aattcctcccatggagccaaacgtccatggatcagggaatcagcatgtgagcagtaggttcagaatcttcacctggaggaaggtcttcctgcacctgcag

SEQ ID NO: 27 - DNA, 100 bp, Artificial sequence
  Synthetic
tctgttcgctcacgcccaactaaggtgcagagggtctaatgccccgattctgagccaacatgggctagatctcagtatcttctcagccatggtttacctt

SEQ ID NO: 28 - DNA, 100 bp, Artificial sequence
  Synthetic
gtgttcagtgtttttcctgcattcattgccacccatcacaattcctcagtgtgcagttcaaataccaggtacaatgaggacactataggcagaagtgagg

SEQ ID NO: 29 - DNA, 100 bp, Artificial sequence
  Synthetic
ataatagggaatgagcagattctaattttaggcctggggacatttcacaaatcccatcagtatgtagaccatgtaagtttgttttggtttgatttgatta

SEQ ID NO: 30 - DNA, 100 bp, Artificial sequence
  Synthetic
gctaaggttttggaaaagttgaagcaacgcgctcagttcctaatcccctccctccagcaggcggagcagagggtttctgttccttgagccccgtgccggc

SEQ ID NO: 31 - DNA, 100 bp, Artificial sequence
  Synthetic
gtattattagagttaggctgataagggtacataaagcacagagcaaaatgctgggtgctccacaatgtacggtaaacaaggaggtatctcccctctggcc

SEQ ID NO: 32 - DNA, 100 bp, Artificial sequence
  Synthetic
caagtctcaagtaggatctatttgagattccaatggcaaatgagacaactaacattccatccctacgaggcatcctgtggtgactttaatttagatcaag

SEQ ID NO: 33 - DNA, 100 bp, Artificial sequence
  Synthetic
ggctgttcatggtaatccaaggaagtggttggagtgaattctacaattctttggcctttaagtcctgaagatgactagttaaataatttgatgcacagac

SEQ ID NO: 34 - DNA, 100 bp, Artificial sequence
  Synthetic
tggcaaaggagaagagacaagtagcatgatccttttaggagaggaatataaatataagtcagcaagaaggaaaaaaaaagatatatcgaggggagaagac

SEQ ID NO: 35 - DNA, 100 bp, Artificial sequence
  Synthetic
cctgggctacggtgagtgttttgattcagaaagggggaaaaaaaaatcttgacttctttgggaaaagaacaaagcgtctgcttgagttactcaaaacgct

SEQ ID NO: 36 - DNA, 100 bp, Artificial sequence
  Synthetic
actgctgctctccagtagggaaatccacctggtaaatctatttccaaccccttacctgggcaggacaagcactttcaaggatgcaatgggttacgcagcc

SEQ ID NO: 37 - DNA, 100 bp, Artificial sequence
  Synthetic
tttcaatatctgattcctttcttttgttccctcttcaccaacccactcctgatgcccaagatctgagcaccagaagagtggagaaggtggtggaagggca

SEQ ID NO: 38 - DNA, 100 bp, Artificial sequence
  Synthetic
aatacagcatagtgtatcaccatgtagtagatgaaggatgtatcagggcaccaggacaccacaagaatatagccagtctacaactgggactgtattattt

SEQ ID NO: 39 - DNA, 100 bp, Artificial sequence
  Synthetic
cagtaatggcagaaatgaagtcatttatagatgctcgttactatgaacacatgcatcttctacagtcatgagtggatttcttacacatatctaatccagt

SEQ ID NO: 40 - DNA, 100 bp, Artificial sequence
  Synthetic
ctgccggccctggagagcgctgttaccaaacaccggtcgtgacccaggacggacagatgtcaccccaatgaagcaagcagaacagaggttggacacaggc

SEQ ID NO: 41 - DNA, 100 bp, Artificial sequence
  Synthetic
ctccgcagccgcggacttttccgcgtctccgaaagggtatttaaatttcatctttaccgcatttctgacagccggaggcagacactgcggcgcgtcccgc

SEQ ID NO: 42 - DNA, 100 bp, Artificial sequence
  Synthetic
gtggtagagtcctgaaacagatcagcaacaaccgcaaatgtgccagccccaggtcttcggacacggaggagaatgacaagaggcgaacacacaacgtctt

SEQ ID NO: 43 - DNA, 100 bp, Artificial sequence
  Synthetic
agtgaactggctttcttactgcagaacctttaatacactgctgtgtcttgtgaatctctcaagagtgaaattcgaatatgctgctttcctgttccttttt

SEQ ID NO: 44 - DNA, 100 bp, Artificial sequence
  Synthetic
agagagagagatgtggtattttggaaagcaccaggcaacagacaaagtagtaactagccttctgtgttaatcaggtacggggctagagggtgggaaggga

SEQ ID NO: 45 - DNA, 100 bp, Artificial sequence
  Synthetic
ggttgttatgataggcatacacatttatttctacacctacatgttttgaaaatcttaagttcacatccatttatccaattccaacataaaaccacatggt

SEQ ID NO: 46 - DNA, 100 bp, Artificial sequence
  Synthetic
tatcaaaggttcaaactatcagataaaatactgatggtggtaaggagtttggaagggggcaggagagagtctttgtattgtaggtaatattcttgatttg

SEQ ID NO: 47 - DNA, 100 bp, Artificial sequence
  Synthetic
ggggggaggaggagaaagaataagagggggaagccacagtaccatttaactcagactaaggtgtaagtgccaatctgcagcaaggcacagcccgcagcca

SEQ ID NO: 48 - DNA, 100 bp, Artificial sequence
  Synthetic
caaaggtcacagaatacataggtaatgcaggaagactgacagtgcggaccgagaggagtctcaaaatcccaaacagagaaacccaggggccacagccagg

SEQ ID NO: 49 - DNA, 100 bp, Artificial sequence
  Synthetic
gcctggcggggcctgttcagaggttgccctggttgcctgagggggtagactggtgtggcttcatgtgatggtgtggacgcaggctgagtgttgtgtcctg

SEQ ID NO: 50 - DNA, 100 bp, Artificial sequence
  Synthetic
tgtgggtttgctgaatatacctgaggatatttgctgagtattccggaggccagaggagtttgggtgggggagggtggttggtgtccttcggtcctccgca